(19) United States
(12) Patent Application Publication
(10) Pub. No.: US 2008/0124416 A1
(43) Pub. Date: May 29, 2008

(54) THERAPEUTIC COMPOSITION FROM GOJI (LYCIUM BARBARUM L.), METHODS OF MAKING AND USING

(75) Inventor: Alex Moffett, Chatsworth, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: RENAISSANCE HERBS, INC., Chatsworth, CA (US)

(21) Appl. No.: 11/945,888

(22) Filed: Nov. 27, 2007

Related U.S. Application Data
(60) Provisional application No. 60/867,575, filed on Nov. 28, 2006.

(51) Int. Cl. A61K 36/815; A61K 31/715; A61P 17/00; A61P 3/10; A61P 9/00

(52) U.S. Cl. 424/777; 514/54

(57) ABSTRACT

The present invention relates to herbal-derived compositions comprising extracts of polysaccharides and carotenoids from Goji species for management of ocular disorders, diabetes, bone metabolism, arthritis, muscle loss, cardiovascular disorders, immunological disorders, fatigue, body weight, sexual disorders, aging disorders, dermatological disorders and neurological disorders. Further provided are methods for making the compositions, and methods for treating subjects with various adverse health conditions with therapeutically or health-promotionally effective amounts of the compositions.

### RELATED APPLICATION

This application claims the benefit of and priority to U.S. Provisional Application Ser. No. 60/867,575, filed Nov. 28, 2006, the contents of which are incorporated by reference herein in its entirety.

### BACKGROUND

1. Field

The present invention relates generally to herbal-derived therapeutic compositions from the Goji species, Lycium barbarum L. More particularly, the present invention relates to herbal compositions that comprise extracts of polysaccharide and carotenoids from Goji species, methods to prepare such compositions, and methods for therapeutic management of health disorders with such compositions.

2. General Background

Goji (Lycium barbarum L.), a cultivated shrub in China producing red berries, has recently come into use as a dietary supplement in the North America. Goji is a well-established agricultural crop in China, and an ample and continued supply may be reasonably anticipated. The Goji berries can also be grown commercially in many other countries as well.

Various uses of Goji in Chinese traditional medicine, whether in the treatment of diabetes, heart disease, sexual insufficiency, or for strengthening bones and muscles and improving vision are of particular interest to aging populations in the West. Pharmacological research of Goji in China, the U.S., and Europe, based on the positive results of earlier studies is being encouraged by the present international interest and financial support for the study of functional foods. As a result, the “anti-aging”, immunological, antidiabetic, antifatigue, and sexual activities of the polysaccharide content are the subject of ongoing research.

Goji has a long history of use in traditional Chinese medicine for helping to maintain and improve vision and for use in the treatment of various ophthalmic conditions. Goji contains a very high content of carotenoids and in particular, a high concentration of the macular carotenoid pigment zeaxanthin. This knowledge coincides with recent laboratory and population-based studies on the prevention and treatment of cataract and age-related macular degeneration indicating that zeaxanthin may provide greater ocular benefits than lutein, a widely known carotenoid found in various supplements for ocular health, including multivitamin formulas.

Goji berries are also a rich food plant source of β-cryptoxanthin, a carotenoid currently being studied for potential use in preventing of age-related bone loss. β-Cryptoxanthin has also has shown antidiabetic activity in animals and in population-based studies is associated with a significant decrease in the risk of developing type 2 diabetes, arthritis, age-related muscle loss, coronary artery disease, and atherosclerosis.

Goji is now widely consumed in the North America in the form of the raw, dried berries and in various beverage products available from numerous web-based outlets, direct sales channels, and retail outlets. Extract powders of Goji have only recently become available in the West. The development of standardized extracts of the berries for encapsulated, liquid, beverage, and other delivery forms is anticipated to favorably increase the market presence of Goji.

Botany and Nomenclature

Native to northern Asia, Lycium barbarum L (family Solanaceae) is a deciduous, woody shrub with thorny branches up to 10 feet in length and bright red edible berries which are the source of the Chinese medicine gou qi or gou qi zi (go-chee-dz) in specific reference to the fruits (Foster and Yue, 1992). English common names include “lycium”, “barbary wolfberry”, and “matrimony vine”, and more recently in the West, “Goji”, a name contrived from the Chinese “gou qi”.

Wild Goji is found at altitudes of 6000 to 9000 feet in sandy soils of the colder provinces of China such as Qinghai, Inner Mongolia, and Shanxi, and is widely cultivated in the northwest province of Ningxia. The species is also found in Iran, India, North Africa, southeastern Europe, the Mediterranean region, and in many parts of the U.S. as an escaped or garden plant.

Traditional Uses of Goji

Traditional Chinese uses of the dried berries (hereafter, “Goji”) are mainly found in the treatment of various ophthalmic conditions, including glaucoma, cataract, and retinitis pigmentosa. Not surprisingly, Goji is utilized in numerous Chinese herbal formulas for ocular conditions and in Chinese culture the dried berries are commonly eaten because of their strongly held reputation of being of benefit to vision. Goji berries are official in the Pharmacopoeia of China for use in the treatment of impaired vision, general debility, anemia, and diabetes and have a long history of use in Chinese medicine for treating “sexual inadequacy”, symptoms of heart disease, and for restoring the strength of bones and muscles.

Nutrients in Goji Berry

In one analysis, the total dietary fiber content of dried Goji berries collected in Ningxia, China and stored for less than a year was 10%, the total fat content was 8.2%, and the protein content was 11.7%—remarkably high for a berry and an indication of a high nutritional value. Others report that after storage for 1-3 years the dried berries from the same province in China contained 0.45% fat, 21% fiber, and 15.6% protein.

Recently published trade books on Goji emphasize the high nutritional content of the berries compared to other fruits and vegetables in the Western diet. However, short of an independent, large sampling study on the macronutrient profile of Goji, the range of nutrient concentrations will not be known for certain. It may be that factors effecting the nutrient profile of plant products such as sunlight, rainfall, soil conditions, processing, storage, and handling are all at work here and we won't know what to expect without further independent analyses. The same factors of variability will undoubtedly apply to carotenoids, vitamins, minerals, and likely amino acids in Goji.

Carotenoids

Goji fruit contains a high amount of total carotenoids (e.g., 269-473 mg/100 g) (Peng et al., 2005). Amounts of β-carotene, well-known as an antioxidant and for its provitamin A activity (Barua, 2004), vary in the dried berries from as little as 1.64-3.64 mg/100 g (Li et al., 1999) to 7.4 mg/100 g in another analysis (Gross et al., 2006); the higher amount being closer to the content in carrots (9.8 mg/100 g) and the lower amounts comparable to those in tomato paste, red pepper, and cantaloupe (1.7, 2.2, and 3 mg/100 g, respectively) (Mangels et al., 1993).

Zeaxanthin

With comparable antioxidant activity in lipids and membranes to that of lutein (Sujak et al., 1999), the structurally very similar oxygenated carotenoid zeaxanthin (a structural isomer of lutein) represents a major antioxidant of Goji. Zeaxanthin inhibits UVB light-induced lipid peroxidation in human lens epithelial (HLE) cells in vitro (Chitchumroonchokchai et al., 2004) and displays in vitro antioxidative activity against A2-PE, a pro-oxidant protein in retinal pigment cells (Kim et al., 2006). In a rat model of hepatic fibrosis, zeaxanthin (12.5 or 25 mg/kg p.o.) significantly increased activities of glutathione-S-transferase and glutathione (antioxidant enzymes), and significantly decreased thiobarbituric acid-reactive substances (TBARS), collagen deposition, 4-hydroxyproline levels, and serum levels of liver enzymes alkaline phosphatase (ALP) and aspartate transaminase (AST) (Kim et al., 2002).

Zeaxanthin and Ocular Health

Studies on carotenoids for the prevention and treatment of cataract and age-related macular degeneration (AMD) have largely concerned dietary lutein or “lutein/zeaxanthin” as undistinguished carotenoids; the lack of separation due to their very similar chemical structure and their recognized collective position as the macular pigment of the human eye. Because Goji contains only small amounts of lutein (5.8 mg/g) (Cheng et al., 2005), studies on the potential benefits of zeaxanthin for ocular health are of particular interest. A number of researchers have recently suggested that zeaxanthin may turn out to be more beneficial to the eyes than lutein, which to date has largely dominated this field of research. Although scientific inquiry is still early, there are a number of reasons to believe that for the prevention or treatment of cataract or AMD, zeaxanthin may hold greater promise than lutein.

Population-based studies in which the effects of the two macular carotenoids are distinguished are scarce; however, in the studies to date, higher versus lower dietary intakes of zeaxanthin alone were associated with significantly reduced risks of macular degeneration and cataracts. A preliminary survey in the U.S. in 380 men and women aged 66-75 years found the risk of AMD in association with higher versus lower plasma levels of lutein or lutein/zeaxanthin was not significant after adjustments for other risk factors; however, for plasma levels of zeaxanthin alone, the association with reduced risk was significant, even after adjustments for antioxidants, other risk factors, and age (Gale et al., 2003).

Zeaxanthin Content and Bioavailability

The dried berries contain the highest known amount of zeaxanthin of any food (82.4 mg/100 g) (Weller and Breithaupt, 2003). In fact, most food plants contain zeaxanthin in only very small amounts (Mangels et al., 1993; Sommenburg et al., 1998). Compared to the richest common food plant sources known, Goji still holds more. For example, the quantities of zeaxanthin in red peppers (16.75 mg/100 g), sea buckthorn berries (3.34 mg/100 g), tangerine juice concentrate (1.3 mg/100 g), sweet corn (0.14 mg/100 g), and persimmons (0.04 mg/100 g) still pale by comparison (Weller and Breithaupt, 2003). Egg yolks, also regarded as an excellent dietary source of zeaxanthin, contain 0.75-1.28 mg/100 g (Schlatterer and Breithaupt, 2006).

Up to 95% of the zeaxanthin content in Goji is zeazanthin dipalmitate, an esterified form (Chitchumroonchokchai and Failla, 2006) with twice the bioavailability in human adults as the nonesterified form, as demonstrated by plasma levels of the carotenoids (Breithaupt et al., 2004). Esterified forms of carotenoids also provide greater stability against oxidative damage than free or nonesterified forms (Biacs et al., 1989). So far, no other food has shown higher contents of zeaxanthin esters than Goji berries (Weller and Breithaupt, 2003).

In a single-blind, placebo-controlled parallel-design trial, healthy adults who consumed 15 grams of the heat-dried berries daily for 28 days showed a 2.5-fold increase in fasting plasma levels of zeaxanthin compared to subjects not ingesting the berries. The dose was calculated to provide approximately 3 mg of dietary zeaxanthin per day (Cheng et al., 2005). In the U.S., the “normal” daily dietary intake of zeaxanthin is estimated to be 0.2-0.375 mg (Bone et al., 2003). Therefore, a daily dosage of 15 grams of Goji supplied 8 to 15 times as much (Cheng et al., 2005). Another estimate places the average daily U.S. intake of zeaxanthin and lutein combined at 1.7-2.3 mg (Anonymous, 2005). Because the intake of lutein is 4-5 times that of zeaxanthin (Bone et al., 2003), 15 grams of the berries per day would therefore supply 1.3-1.7 times the intake of both carotenoids combined.

β-Cryptoxanthin

The dried berries are a rich source of the carotenoid, β-Cryptoxanthin (8.58-13 mg/100 g) (Li et al., 1999). According to the USDA National Nutrient Database, highest amounts of β-Cryptoxanthin in any fruit or vegetable are 2.07 mg/100 g in red peppers and 1.45 mg/100 g in pumpkin (USDA National Nutrient Database). Goji may therefore be the highest food plant source of β-Cryptoxanthin known. Although found in the berries in esterified form, as β-Cryptoxanthinpalmitate (Li et al., 1999), human bioavailability of esterified and free forms of the carotenoid are comparable (Breithaupt et al., 2003).

β-Cryptoxanthin and Diabetes

The use of Goji to treat diabetes in traditional Chinese (Tu et al., 1992) has recently begun to receive the attention of research pharmacologists. For example, a decoction of dried berries administered to rabbits with alloxan-induced diabetes (250 mg/kg p.o. per day for 10 days) significantly decreased blood glucose levels, significantly improved glucose tolerance levels, and while significantly decreasing total serum cholesterol levels, serum HDL-c levels were significantly increased (Luo et al., 2004).

Among the candidate constituents contributing to the antidiabetic activity of the berries is b-cryptoxanthin. Oral administration of the carotenoid (50 or 100 μg/kg/day for 14 days) in a rat model of diabetes (streptozotocin-induced) was found to significantly attenuate symptoms of the condition, including raised serum levels of glucose, triglycerides, and calcium, elevated body weight, and bone loss (Uchiyama and Yamaguchi, 2005).

β-Cryptoxanthin and Bone Metabolism

A growing body of evidence, including clinical, observational, and experimental studies, now indicates that bone health and the consumption of fruit and vegetables are strongly linked (Lanham-New, 2006). With osteoporosis on the rise, the burning question is what are the contributing active principles? The traditional Chinese use of Goji berries for strengthening the bones (Huang, 1999) recently received scientific support when a water extract of dried berries was found to significantly stimulate the in vitro proliferation of osteoblast-like (UMR106) cells. Curiously, an alcohol extract was not effective (Li et al., 2001), which suggests that water-insoluble constituents such as carotenoids are not involved. However, activity-guided isolation of the constituents responsible for the effect remain to be conducted and the insignificant activity of the alcohol extract in a single assay does not mean it will lack activity in others.

Among a number of phytochemicals currently being examined for the prevention or therapy of bone loss associated with age, recent evidence suggests that β-cryptoxanthin may become a subject of ongoing interest. In vitro studies have shown that in micromole amounts, β-cryptoxanthin significantly increases the alkaline phosphatase activity (indicating the stimulation of bone formation) and calcium content of cultured bone cells from the femoral tissues of young rats (Yamaguchi and Uchiyama, 2003); potently inhibits osteoblast-like cell formation (a process related to bone resorption) induced by a number bone-resorbing factors (e.g., tumor necrosis factor-β and protein kinase C) in mouse marrow cultures (Uchiyama and Yamaguchi, 2004); directly stimulates bone formation in rat cortical and trabecular bone tissues; increases their calcium content, DNA content, and alkaline phosphatase activity; completely inhibits the decrease in calcium content induced by bone-resorbing factors, prostaglandin E2 and parathyroid hormone (Yamaguchi and Uchiyama, 2004); and stimulates transcriptional activity, proliferation (Uchiyama and Yamaguchi, 2005a), cell differentiation, and mineralization of cells related to bone formation (osteoblastic MC3T3-E1 cells) (Uchiyama and Yamaguchi, 2005b).

β-Cryptoxanthin and Arthritis

A prospective population-based study (Pattison et al., 2005) reported that orange juice may contribute to the prevention of arthritis. The study set out to determine whether dietary carotenoids would show any association with a reduced risk of developing inflammatory polyarthritis. The study was predicated on the findings of others who showed that serum antioxidant levels may play a role in the inflammation of rheumatoid arthritis and protect against its development. The 8-year study followed the dietary intake of carotenoids in 88 subjects aged 45-75 years while checking for signs of inflammatory polyarthritis, defined as synovitis affecting at two or more joints. Results were compared to those of 176 healthy controls matched for age and gender. When those with the lowest dietary intakes of β-cryptoxanthin were compared to those with the highest intakes (>365 mg/day), the carotenoid was associated with a reduced risk of developing the condition of about 58%. The association was still significant after adjustments for other factors including smoking. Median intakes of β-cryptoxanthin were 40% lower in those who developed inflammatory polyarthritis compared to those who did not. Although not as strongly associated, the only other dietary carotenoid in the study that showed any significant association with a reduced risk of developing the condition was zeaxanthin. A 20% lower intake of zeaxanthin was enough to show an association with having the condition. The higher dietary intake of zeaxanthin associated with the reduced risk amounted to >39 mg/day; however, the association was of borderline significance. Although the dietary sources of zeaxanthin were not described, the intake of β-cryptoxanthin was mainly from consuming oranges, Satsumas, and orange juice. The authors concluded that the daily equivalent of a single glass of freshly squeezed orange juice provided enough of the carotenoid to reduce the risk of developing inflammatory polyarthritis (Pattison et al., 2005).

β-Cryptoxanthin and Muscle Loss

Sarcopenia or loss of muscle mass is another possible condition suggested in the traditional uses of the berries for restoring the strength of bones and muscles (Huang, 1999). Sarcopenia appears to be a largely age-related decrease in skeletal muscle mass caused by an age-related decrease in muscle power and strength. Because oxidative stress may play a role in sarcopenia, an epidemiological study on plasma levels of carotenoids and vitamin E was conducted to see whether dietary carotenoids or the vitamin might be associated. The subjects of the study were 669 women aged 70-79 with various degrees of ability, ranging from nondisabled to severely disabled. Along with plasma levels of the antioxidants, knee, hip, and grip strength were measured. The data showed that the risk of lower strength of the knee, grip, or hip was reduced in association with higher plasma levels of b-cryptoxanthin, lutein/zeaxanthin, a-carotene, and b-carotene. The association held after adjustments for potential confounders, including race, age, arthritis, cardiovascular disease, plasma concentrations of interleukin-6, and smoking. For vitamin E, higher versus lower plasma levels were associated only with greater grip strength and knee strength (Semba et al., 2003).

β-Cryptoxanthin and Cardiovascular Disease

In a study of 50 Swedish patients with coronary artery disease (CAD), lower plasma levels of β-cryptoxanthin were found to be significantly correlated with CAD compared to healthy controls (p<0.05) and the correlation with lower levels of lutein and zeaxanthin held even greater significance (p<0.001). The association was also significant for serum levels of β-carotene (p<0.05), but not for a-carotene or lycopene (Lidebjar et al., 2006). In the U.S., a study on serum levels of antioxidants was examined in 477 subjects of either sex aged 40-60 years without signs or symptoms of CAD. Atherosclerosis was measured using ultrasound exams of intima-carotid thickness (IMT) at the start and 18 months later. Progression of IMT was significantly reduced in those with higher baseline levels of serum β-cryptoxanthin (p=0.015), lutein (p=0.017), a-carotene (p=0.003), and zeaxanthin (p=0.0004), but not other carotenoids, ascorbic acid, or tocopherols (Dwyer et al., 2004). In a similar study in 462 subjects aged 53-65 years in the U.S., a reduced development of atherosclerosis as measured by IMT 1-6 years after entry was significantly associated with higher compared to lower serum levels of β-cryptoxanthin and lutein/zeaxanthin (Iribarren et al., 1997).

Antioxidant Constituents

Against free radical generation (hypoxia-induced) in male mice, an aqueous suspension of Goji (0.5 mL of a 20% concentrate per day for 16 days) produced significant increases in SOD (superoxide dismutase) and catalase activity in the heart muscle, liver, and lung tissue, and the total antioxidant capacity (Li et al., 2002). In vitro studies have shown that a water extract of the dried berries exhibits dose-dependent antioxidant activity with superoxide radical-scavenging and formation-inhibiting activity and anti-lipid peroxidation activity (malondialdehyde production in rat liver homogenate). From extract concentrations of 0.5-5 mg/mL, malondialdehyde (MDA) production was inhibited by 22% to 70.1%. At concentrations of 0.1-10 mg/mL, superoxide was inhibited (scavenged) by 35.1% to 82.2% and the formation of superoxide anions (xanthine oxidation inhibition test) was inhibited by 40.7% to 88.5% (Wu et al., 2004). However, in the ORAC assay (oxygen radical antioxidant capacity assay) a methanol extract of the fruit produced a greater antioxidant value than a water extract (Luo et al., 2004).

Polysaccharides (Glycoconjugates)

The majority of studies on Goji concern the polysaccharide or “glycoconjugate” fraction. Including various more or less characterized individual polysaccharides, research has shown that they provide a wide range of pharmacological activities.

Immunological Activity

In vitro studies have shown that the polysaccharide fraction of the berries stimulates interleukin-2 production in the splenocytes of adult mice and in spleen cells from aged mice, restores the activity to that of adult mice (Geng et al., 1989). The crude polysaccharide fraction of the fruit was also shown to enhance splenocyte production of immunoglobulin-G in senescence-accelerated mice following intragastric administration (Qi et al., 2001). For the most part, however, activity demonstrated by the oral route of administration has been lacking. More recently, a purified polysaccharide fraction of the fruit (“LBP3p”), described as a polysaccharide-protein complex, was shown to produce significant tumor-inhibiting activity in Sarcoma 180-bearing mice. Besides decreasing lipid peroxidation, the polysaccharide stimulated macrophage phagocytosis, antibody secretion of spleen cells, cytotoxic T lymphocyte (CTL) activity, spleen lymphocyte proliferation, and interleukin-2 expression. The most effective dose was 10 mg/kg p.o. (Gan et al., 2004).

Effects on Fatigue and Body Weight

Studies to date suggest that the traditional use of Goji in Chinese medicine against general debility (Tu et al., 1992) and strengthening muscles (Huang, 1999) may be due in some measure to the activity of the polysaccharide content. Administration of a crude polysaccharide fraction of the berries in male mice (10 mg/kg i.g.) significantly enhanced their recovery from fatigue following exercise. The effect was evident in their significantly enhanced liver and muscle glycogen reserves, increased lactate dehydrogenase activity prior to swimming and 90 and 150 minutes after, and decreased blood urea nitrogen (BUN) content following strenuous exercise. Lactate dehydrogenase converts lactic acid which accumulates in muscle during exercise to pyruvic acid, thereby reducing lactic acid levels in the muscles. BUN is a product of metabolism during exercise and can be used to measure the body's tolerance to physical loads. The greater the increase in BUN levels, the less the body has adapted to physical loads. In tired mice after exercise, the polysaccharide fraction significantly accelerated the rate of clearance of BUN, thereby allowing a faster recovery from fatigue. The results suggest that the polysaccharide fraction of the berries enhances tolerance and increases adaptability to loads while at the same time it retards fatigue. Overall, however, better results were achieved using a purified polysaccharide fraction (“LBP-X”). After tests using a range of dosages (5-100 mg/kg), 10 mg/kg produced the best results (Luo et al., 1999). Both the antifatigue effects and the optimum dosage of 10 mg/kg i.g. were confirmed in a follow-up study in mice treated only with the purified fraction, LBP-X. LBP-X is described as readily soluble in water and relatively free from the large amounts of pigments found in the crude polysaccharide fraction (Luo et al., 2000).

Antidiabetic Activity

The hypoglycemic activity of orally administered extracts and derivatives of the dried fruits was tested in alloxan-induced diabetes in rabbits of either sex. The effects of a purified, crude polysaccharide (glycoconjugate) fraction (“crude LBP”) and “LBP-X”, the major fraction (each at 10 mg/kg/day for 10 days), were compared to those of a water decoction of the dried fruits (250 mg/kg/day). LBP-X was found to be composed of 6 monosaccharides (largely rhamnose and galactose) and 8.46% amino acids of which 17 were identified. Whereas each of the preparations produced equally significant decreases in blood glucose levels, the decrease from the decoction (8.04±2.51) and crude LBP (8.47±2.96) was less than that of LBP-X (14.13±6.35). From glucose tolerance tests performed prior to inducing diabetes, after diabetes, and before treatment, and again after 10 days of treatment, the glucose tolerance curves showed that although the extracts and fractions derived from the berries improved tolerance, they failed to normalize it. An equally significant improvement in glucose tolerance levels at the various time points were found from the water decoction and crude LBP (each p<0.05) whereas the effect was of greater significance from LBP-X (p<0.01) (Luo et al., 2004).

Against the hyperlipidemia-induced by alloxan in the same rabbits, a greater decrease in total serum cholesterol (TC) levels (54.1%) and a greater increase in HDL-c (136.8%) was found after oral treatment for 10 days with crude LBP than from either LBP-X or the water decoction of the dried berries. Even so, both the decrease in TC and the increase in HDL-c from LBP-X (41.4% and 39.7%, respectively) and from the decoction (51.8% and 57.4%, respectively) was significant. However, the decrease in triglyceride levels obtained from the decoction of the berries was nearly double that of the polysaccharides (71.2% vs 36.3% and 38.3% from crude LBP and LBP-X, respectively) (Luo et al. 2004).

Anti-Aging Effects

In testing the polysaccharide fraction of the berries for potential “anti-aging” effects, researchers used a model of accelerated aging (D-galactose-induced) in a strain of female mice. Administered intragastrically (100 mg/kg/day) for 8 weeks, the fraction produced a number of anti-aging effects: significantly reducing serum levels of advanced glycation end-products; inhibiting the decline in immune response (increasing lymphocyte proliferation and interleukin-2 production); inhibiting the decrease in spontaneous motor activity and learning and memory abilities; preventing the decrease in red blood cell SOD activity; and preventing the increase in skin hydroxproline content (Deng et al., 2003).

Sexual and Hormonal Effects

Goji berries are said to be used in the majority of Chinese herbal formulas for promoting fertility (Luo et al., 2006). Use of the fruits in India as an “aphrodisiac” (Jain and DeFilipps, 1991 op cit) and in China for “sexual inadequacy” (Leung, 1995 op cit) would suggest that these traditional uses may have a pharmacological basis.

Using a purified polysaccharide fraction (oligosaccharides removed) of the dried berries (“LBP”), effects on the sexual behavior and reproductive function were investigated in a hemicastrated (right testis removed) rats. Those administered LBP (10 mg/kg/day for 21 days) showed significantly shortened latency of penis erection, mount latency with female rats, and greater percentage of mounting compared to a hemicastrated negative control group treated with saline. Compared to a positive hemicastrated control group treated with testosterone propionate (2 mg/kg subcutaneously per day), the results in the LBP group were not significantly different, nor compared to a control group of normal, uncastrated rats treated with saline (10 mg/kg p.o per day). As for changes in hormone levels, luteinizing hormone and follicle stimulating hormone levels showed no significant changes in any of the groups, whereas testosterone levels significantly increased and estradiol levels significantly decreased in all but the saline-treated hemicastrated rats (Luo et al., 2006).

Dermatological Effects

Traditional use of the berries for improving the complexion (Foster and Yue, 1992) has so far received little attention. As noted earlier, when the potential “anti-aging effect” of the polysaccharide fraction (100 mg/kg i.g. per day for 8 weeks) was tested in an animal model of accelerated aging (D-galactose-induced in female C57BL/6J mice), the treatment significantly inhibited (p<0.01) the increase in skin hydroxproline content, an indirect marker for the collagen content of the skin (Deng et al., 2003).

As for what constituents might be responsible for the increased skin hydroxyproline content, gylcoconjugates may be suspected. In vitro studies found that a low molecular weight (23.7 kDa) glycoconjugate derivative from the dried berries (LbGp5; yield 0.45 mg/g) could inhibit the decrease in type-I collagen in human dermal fibroblast cultures under suboptimal conditions and that a glycoconjugate fraction (“LbGp”; 8 mg/g dried fruit) inhibited the increase of MMP-1 (matrix metalloproteinase-1), a marker of skin cancer and premature aging of the skin (Zhao, Alexeev et al., 2005).

Neurological Research

Inspired by the reputation of Goji as an “anti-aging” food and some previous studies suggesting an “anti-aging” function of the berries in animal models, researchers in China have recently begun to investigate Goji for effects on neurological cells. For example, it was recently found that a water extract of the berries (deproteinized) exhibits dose-dependent in vitro protective activity against the neurotoxic effects of plaque-forming cells (Ab peptides) associated with Alzheimer's disease (Yu et al., 2006). Research on the in vitro neuroprotective effects of a standardized extract of Goji (uncharacterized) against a well-known stress-inducer (dithiothreitol) showed that, independent of antioxidant activity, the extract exhibits significant cytoprotective effects against cell death of cortical neurons (Yu et al., 2005). While still early in its development, research on the potential neuroprotective effects of Goji will undoubtedly continue.

Human Studies

In contemporary Chinese traditional medicine, the daily dosage of the dried fruits is 5-18 g (Yen, 1992; Tu et al., 1992; Bensky and Gamble, 1993) and larger, softer, sweeter berries, red in color and with fewer seeds, are considered the best (Yen, 1992). In clinical studies, however, the dosages are typically higher.

In 27 healthy adult students (ages 18-25 years) in China who consumed 50 g of the dried berries per day for 34 days, an open-label study (without a placebo control or double-blinding) with a controlled diet containing no vitamin A found that compared to baseline, serum vitamin A levels in deficient subjects increased to an adequate range, β-carotene levels significantly increased in all subjects, the ability to see in poorly lighted areas (night blindness) was significantly improved, and for all but 7 of the subjects, the physiological blind-spot area significantly normalized; the latter an indication of pre-existing vitamin A deficiency. Accordingly, it was calculated that about 19% of the subjects had vitamin A deficiency (Shen et al., 1990). Among the provitamin A carotenoids identified in the berries so far (Li et al., 1999), β-carotene and β-cryptoxanthin (hydroxy-β-carotene), estimated to have about half the provitamin A activity of β-carotene (Barua, 2004), would be largely responsible for increasing serum vitamin A levels and likely the resultant improvement in night blindness.

An open-label study in China on the effects of Goji in 25 randomly selected healthy volunteers (ages 64-80 years) of either sex found that after 10 days of consuming the dried berries (50 g/per day), blood levels of hemoglobin and superoxide dismutase (SOD) were significantly increased and lipid peroxide levels were significantly decreased. SOD levels increased by 48%, hemoglobin increased by 12%, and the lipid peroxide content of their blood decreased by 65%. The authors noted that because with age SOD levels decline while lipid peroxide levels rise, the results suggest that the berries have an “anti-aging function” (Li et al., 1991).

An open-label study on physiological and immunological functions of people aged 60 and over found that after 10 days of consuming Goji (50 g/day), the subjects showed significant increases compared to baseline in values of immunoglobulin G, immuglobulin A, the responsiveness of lymphocytes (Stimulation Index), levels of cyclic AMP, and testosterone. Researchers concluded that the berries may therefore provide health benefits to those in old age (Xiao and Chen, 1988).

Another open-label study in healthy adult volunteers (males) administered Goji at a dosage of 50 g/day for 10 days found their leukocyte counts significantly increased compared to baseline and that there were no obvious side effects from the treatment (Xiao and Chen, 1988). Using the same dosage in an open-label study in 27 cancer patients receiving chemotherapy and radiotherapy, leukocyte counts depressed from the cancer therapies were significantly increased by the berries (Xiao and Chen, 1988).

Toxicology

No signs of toxicity were found in organs, blood biochemistry, behavior, and other parameters from daily intragastric administration of a water extract of the berries of a variety of the plant “Ningxia gou qi” grown in the province of Hebei (Zhang et al., 1998). Since the common name is specific to Lycium barbarum L. (McGuffin et al., 2000) and to L. barbarum var. barbarum (Flora of China, 1994), it is presumed that they used the berries of the latter. The extract was administered at daily intragastric dosages of 20 g/kg and 30 g/kg in Sprague-Dawley rats of either sex for 28 days. Evidence of immunopotentiation was found in significant increases in the total leucocyte number, monocyte and lymphocyte rates, and thoracic gland weight. During the two-week observation period in 8 rats after the extract was withdrawn, only the lymphocyte and monocyte counts remained significantly higher compared to the control group (Zhang et al., 1998).

Drug Interactions

One suspected case of a drug interaction with a concentrated “tea” made from Goji berries was reported in the U.S. The case concerned a Chinese woman aged 61 years who consumed the berries in the form of a juice while stabilized on warfarin therapy. She remained stabilized until she drank a cup of the concentrate 3-4 times per day for 4 days. It was calculated that she consumed approximately 2.1 grams of the juice solids per day. Although her blood coagulation index dramatically increased after drinking the “tea” and she was eventually stabilized after discontinuing the beverage and adjusting her dosage of warfarin, she showed no signs or symptoms of bleeding or abnormal bruising. Besides warfarin, medications taken by the patient on a daily basis were benazepril (40 mg), digoxin (0.25 mg), atenolol (25 mg), and fluvastatin (20 mg). Her medical conditions were tricuspid regurgitation, hypertension, and hyper-cholesterolemia and she had a history of recurring atrial fibrillation. An in vitro test of the juice concentrate for inhibition of drug metabolism on cytochrome P450 using human liver microsomes found a positive result with the most potent enantiomer of warfarin (S-warfarin). However, the value obtained indicated that the concentrations required to produce inhibitory activity would be difficult to achieve in vivo. The authors could not rule out a direct anticoagulant effect of the tea concentrate, effects on absorption of warfarin, or effects on other systems of drug metabolism such as p-glycoprotein (Lam et al., 2001).

Allergic Reactions

Allergy to the berries appears to be rare. Only two cases were found in the literature where subjects experienced an itchy rash from consuming the berries. In both cases the allergy was confirmed when the patients were challenged by having them eat a second equal dose of the berries. The first case was a man aged 23 who ate 20 g of the berries (Yang, 1985) and the second was a woman aged 59 who consumed 30 g of the washed and simmered berries (Ding and Wang, 1994). It follows that individuals with an allergy to plants of the nightshade family (Solanaceae) may also be allergic to Goji berries.

Background References
Leung A Y, Foster S. Encyclopedia of Common Natural Ingredients. Second ed. New York, N.Y.: John Wiley and Sons, Inc; 1996:358-61.
Tu G, et al., eds. Pharmacopoeia of the People's Republic of China. Guangzhou, China: Guangdong Science and Technology Press; 1992:75-6.
Huang K C. The Pharmacology of Chinese Herbs. Second ed. Boca Raton, Fla.: CRC Press; 1999:269.
Leung A Y. Better Health with (Mostly) Chinese Herbs and Food. Glen Rock, N.J.: AYSL Corporation, 1995:60-62.
Jain S K, DeFilipps R A. Medicinal Plants of India. Vol. 2. Algonac, Mich.: Reference Publications; 1991:565.
Peng Y, Ma C, Li Y, Leung K S, Jiang Z, Zhao Z. Quantification of zeaxanthin dipalmitate and total carotenoids in Lycium fruits (Fructus Lycii). Plant Foods Hum Nutr. 2005;60(4):161-164.
Leung I Y F, et al. Absorption and tissue distribution of zeaxanthin and lutein in rhesus monkeys after taking fructus lycii (gou qi zi) extract. Invest Opthalmol Vis Sci. 2001;42:466-71.
Cheng C Y, et al. Fasting plasma zeaxanthin response to Fructus barbarum L. (wolfberry; Kei Tze) in a food-based human supplementation trial. Br J Nutr. 2005;93:123-30.
Weller P, Breithaupt D E. Identification and quantification of zeaxanthin esters in plants using liquid chromatography-mass spectrometry. J Agric Food Chem. 2003;51:7044-9.
Zhou L, et al. The identification of dipalmityl zeaxanthin as the major carotenoid in gou qi zi by high pressure liquid chromatography and mass spectrometry. J Ocul Pharmacol Ther. 1999;15:557-65.
Biacs P A, et al. Studies on the carotenoid pigments of paprika (Capsicum annuum L. var. Sz-20). J Agric Food Chem. 1989;37:350-3.
Chitchumroonchokchai C, Failla M L. Hydrolysis of zeazanthin esters by carboxyl ester lipase during digestion facilitates micellarization and uptake of the xanthophylls by caco-2 human intestinal cells. J Nutr. 2006;136:588-94.
Breithaupt D E, et al. Comparison of plasma responses in human subjects after the ingestion of 3R,3R′-zeaxanthin dipalmitate from wolfberry (Lycium barbarum) and non-esterified 3R,3R′-zeaxanthin using chiral high-performance liquid chromatography. Br J Nutr. 2004;91:707-13.
Bowen P E, et al. Esterfication does not impair lutein bioavailability in humans. J Nutr. 2002;132:3668-73.

### SUMMARY

For example and not by way of limitation, certain embodiments of the present invention provide therapeutic or health-promoting herbal compositions from Goji species, typically from the berries of the plant, and typically from the species Lycium barbarum. Other embodiments of the present invention provide a method for the extraction of therapeutically active polysaccharides and carotenoids from Goji species. Still other embodiments of the present invention provide methods of delivering a therapeutically effective dosage of both polysaccharides and carotenoids to subjects whose medical condition indicates that they would benefit from such treatment.

Embodiments of the invention typically provide polysaccharides in the form of glycoconjugates, such as conjugates with neutral sugars, minerals, proteins, and uronic acid. Embodiments of the invention include all carotenoids that may be derived from Goji, such as, for example, of zeaxanthin, zeaxanthin dipalmitate, β-cryptoxanthin, β-carotene, and lutein. The respective concentrations of polysaccharides and carotenoids in the inventive compositions are independently controlled by the process of manufacturing. Thus, the concentrations of these two major chemical classes in the compositions do not necessarily covary with respect to each other, nor are the final concentrations necessarily reflective of their original relative concentrations in the source material.

Embodiments of the present invention further provide methods of preparing herbal compositions for therapeutic use comprising extracts of polysaccharides and carotenoids from Goji species; such methods may further include incorporation of pharmaceutically acceptable additives. Briefly, embodiments of the inventive process for the preparation of an herbal composition include steps of hot water extraction of Goji berry fruit to obtain polysaccharide extract, extraction of previously water-extracted spent with alcohol, followed by ethyl acetate extraction, typically under reflux conditions, to obtain carotenoid extract, and combining the various extracts into compositions such that the carotenoid and polysaccharide concentrations are independently controlled.

Other embodiments of the present invention provide methods for the use of the therapeutic or health-promoting herbal composition for management of particular disorders or health conditions, such as, for example, ocular disorders, diabetes, bone metabolism, arthritis, muscle loss, cardiovascular disorders, immunological disorders, fatigue, body weight, sexual disorders, aging disorders, dermatological disorders and neurological disorders in subjects in need thereof. Many conditions that could be treated or alleviated are discussed in the background section. In embodiments of the invention that involve treatment of subjects, a therapeutically or healthfully effective amount of the composition is administered to a subject with such disorders or adverse health conditions. A therapeutically effective dosage is one that provides relief of either the symptoms of such disorders, or provides a therapeutic correction of the physiological or pathological factors underlying the disorder.

A therapeutic dosage may also be applied prophylactically, in which case the therapeutic benefit is to prevent the onset of such disorders, or attenuate their severity or duration. Methods of use of embodiments of the present invention, in addition to therapeutic, may also, further, or more fundamentally be health-promoting, i.e., healthful or salutory for the subject being administered or ingesting embodiments of these inventive Goji-derived compositions. In embodiments that provide a healthful benefit, such benefit is provided by an healthfully-effective dosage. In some embodiments, the beneficial effect of Goji-derived compounds may be plainly nutritional in nature, as for example the effects of the carotenoid, lutein, with respect to the management of ocular disorders.

Some embodiments of the invention may further include other components or second agents. By “second agent(s)” or “second active agent” is meant any active ingredient beyond the Goji-derived carotenoids or polysaccharides, which may thereby be referred to as the “first agents”. Such second agents may provide therapeutic or healthful benefits of their own, in addition to the therapeutic or healthful benefits provided by the first agents. Such second agents may include compounds that are nutritional; some compounds may provide beneficial effects at very low daily dosages, in which case they may be referred to as micronutritional ingredients. When second agents provide a nutritional or micronutritional benefit, they may be referred to more specifically as “nutritional agents”. The combined effects of the first agents and second agents may be additive or they may be synergistic with respect to the benefits of each individual active agent or ingredient.

The Goji-derived polysaccharides and carotenoids, in some embodiments, may have separate therapeutic mechanisms of action. The combined therapeutic or healthful effects of the two chemical classes may in some cases be additive with respect to the benefits derived from each class alone, or in some cases, the benefits may be synergistic, creating a total therapeutic effect greater than the sum of the individual benefits of each chemical class.

### DETAILED DESCRIPTION

The present invention is in relation to a an herbal composition for therapeutic or health-promoting use comprising extracts of polysaccharides and carotenoids from Goji species optionally along with second active agents and/or pharmaceutically acceptable additives. Certain embodiments of the inventive compositions are detailed below.

One embodiment of the present invention includes compositions of extracts that are prepared from the berries of Goji species.

In some embodiments of the present invention, the polysaccharides are at a concentration ranging from 10%-80% w/w.

In some embodiments of the present invention, the carotenoids are at a concentration ranging from 0.1%-80% w/w.

Some embodiments of the present invention include the polysaccharides derived from Goji, without the carotenoids; other embodiments include the carotenoids derived from Goji, without the polysaccharides.

In some embodiments of the present invention, the inventive composition delivers a dosage of polysaccharides and carotenoids to that is sufficient to provide a healthful or therapeutic benefit.

In some embodiments of the present invention, the polysaccharides are glycoconjugates, being conjugated with moieties that may include any one or more of neutral sugars, minerals, proteins and uronic acid. In some embodiments, the respective final concentrations of specific glycoconjugated polysaccharides may be individually modulated. By increasing or decreasing specific glycoconjugated polysaccharides, the relative concentrations of each compound in the final composition with respect to the concentrations in the Goji source material will vary from compound to compound. The purpose of such specific modulation of particular polysaccharides is to conform the composition to be appropriate for achieving particular healthful or therapeutic effects.

In some embodiments of the present invention, the carotenoids include any one or more of zeaxanthin, zeaxanthin dipalmitate β-cryptoxanthin, β-carotene, and lutein. In some embodiments, the respective final concentrations of specific carotenoids may be individually modulated. By increasing or decreasing specific carotenoids, the relative concentrations of each compound in the final composition with respect to the concentrations in the Goji source material will vary from compound to compound. The purpose of such specific modulation of particular polysaccharides is to conform the composition to be appropriate for achieving particular healthful or therapeutic effects.

Embodiments of the present invention relate to a process for the preparation of an herbal composition comprising steps of hot water extraction of Goji berry fruit to obtain polysaccharide extract, extraction of water extracted spent with alcohol followed by ethyl acetate under reflux conditions to obtain carotenoid extract and combining both the extracts optionally along with pharmaceutically acceptable additives. In some embodiments, the healthful beneficial effect of the Goji-derived compounds may be considered nutritional in nature. Lutein, for example, may provide nutritional benefit for various ocular disorders, in which it serves as a visual pigment, particularly in the macula of the eye. In some embodiments, the Goji-derived compositions may further include other ingredients, which may, for example, be nutrients or micronutrients, that provide additional therapeutic or healthful benefit.

The present invention further relates to the therapeutic or health-promoting use of an herbal composition comprising extracts of polysaccharides and carotenoids from Goji species, optionally including pharmaceutically acceptable additives, to manufacture a medicament for management of ocular disorders, diabetes, bone metabolism, arthritis, muscle loss, cardiovascular disorders, immunological disorders, fatigue and body weight, sexual disorders, aging disorders, dermatological disorders and neurological disorders in a subject in need thereof.

The technology of the application is further described with the help of the following examples, which, however, should not be construed to limit the scope of the invention. Specific volumes and numbers of extraction procedures are provided merely by way of example, a practitioner will recognize and implement variations in volumes, both relative and absolute, temperature, and time, that are included in the scope of the invention.

### EXAMPLES

### Process Description of Goji Berry Extract

1. Process for Extraction of Total Polysaccharides (Product # 1)

Dried Goji berry fruits are extracted with water at a temperature of about 90-95° C. Typically, there are three extractions, using five volumes of solvent relative to the volume of the berries. All the extracts are then combined to yield “Extract #1” and a spent material. Extract 1 is then filtered, concentrated, and dried, to yield “Product # 1”. Product #1 contains the polysaccharides from the Goji fruit; the concentration of total polysaccharides may be adjusted to a concentration varying between about 10% w/w to about 80% w/w.

2. Process for Extraction of Total Carotenoids (Product # 2)

The spent Goji berry material is further extracted with about 8 volumes of 35% alcohol under reflux conditions, and then cooled, and filtered to remove the solvent. The alcohol fraction may be considered a “first-carotenoid-containing extract”. The spent material is further extracted, in this instance, with about 8 volumes of ethyl acetate under reflux conditions. The ethyl acetate fraction may be considered a “second carotenoid-containing extract”. Typically, there are a total three such ethyl acetate extractions, which may be combined together. All three combined extracts (from water, from alcohol, and from ethyl acetate) are then combined, filtered, and concentrated to about 25%-about 30% total solids, to yield “Extract #2”.

A half volume of 35% alcohol is then added to Extract #2, which is then stirred for about 1 hour. The precipitate is next collected as a wet cake. The wet cake is then washed with acetone at room temperature, and dried under vacuum at a temperature in the range of about 50°-55° C., to yield “Product #2”. Product #2 contains the carotenoids of the Goji fruit; the concentration of total carotenoids may be adjusted to a concentration varying between about 0.10% w/w to about 80% w/w.

3: Process for Preparation of a Goji-Based Herbal Composition (Product # 3)

Product #1 and Product #2 are then combined to yield Product #3, in which the total polysaccharides are present at a concentration varying between about 10% w/w to about 60% w/w, and the total carotenoids are present at a concentration varying between about 0.10% w/w to about 20% w/w. The concentrations of the polysaccharides and the carotenoids in the final composition are each at a given concentration relative to the concentration in the source Goji material. The relative respective concentrations of the polysaccharides and the carotenes, and the respective final concentrations in absolute terms, are determined by inventive process methods that allow formulation of compositions with the respective polysaccharide and carotenoid concentrations that vary independently of each other.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A therapeutic composition comprising polysaccharides and carotenoids extracted from Goji species.

2. The composition of claim 1, wherein the polysaccharides and carotenoids are extracted from the berries of Goji species.

3. The composition of claim 1, wherein the polysaccharides are present at a concentration ranging from about 10%-about 80% w/w.

4. The composition of claim 1, wherein the carotenoids are present at a concentration ranging from about 0.10%-about 80% w/w.

5. The composition of claim 1, wherein the polysaccharides and carotenoids are each at a given final concentration, the respective final concentrations of the polysaccharides and the carotenoids being determined independently of each other.

6. The composition of claim 1, wherein the polysaccharides are conjugated with moieties such that they are glycoconjugated polysaccharides.

7. The composition of claim 6, wherein the moiety with which the polysaccharides are conjugated is selected from a group consisting of neutral sugars, minerals, proteins, and uronic acid.

8. The composition of claim 7, wherein the final concentration of any of the glycoconjugated polysaccharides is determined independently of the other glycoconjugated polysaccharides.

9. The composition of claim 1, wherein the carotenoids include at least one of zeaxanthin, zeaxanthin dipalmitate, β-cryptoxanthin, β-carotene, and lutein.

10. The composition of claim 9, wherein the final concentration of any of the carotenoids is determined independently of the other carotenoids.

11. The therapeutic composition of claim 1, further comprising at least one nutritional agent.

12. A therapeutic composition comprising polysaccharides extracted from Goji species.

13. The composition of claim 12, wherein the polysaccharides are present at a concentration ranging from about 10%-about 80% w/w.

14. The composition of claim 12, wherein the polysaccharides are conjugated with moieties such that they are glycoconjugated polysaccharides.

15. The composition of claim 14, wherein the moiety with which the polysaccharides are conjugated is selected from a group consisting of neutral sugars, minerals, proteins, and uronic acid.

16. The composition of claim 14, wherein the final concentration of any of the glycoconjugated polysaccharides is determined independently of the other glycoconjugated polysaccharides.

17. The therapeutic composition of claim 12, further comprising at least one nutritional agent.

18. A therapeutic composition comprising carotenoids extracted from Goji species.

19. The composition of claim 18, wherein the carotenoids are present at a concentration ranging from about 0.10%-about 80% w/w.

20. The composition of claim 18, wherein the carotenoids are selected from a group consisting of zeaxanthin, zeaxanthin dipalmitate, β-cryptoxanthin, β-carotene, and lutein.

21. A method of preparing of a therapeutic composition from Goji species, comprising:
extracting Goji berries with hot water to obtain a polysaccharide-containing extract and a spent material;
extracting the spent material with alcohol to obtain a first carotenoid-containing extract;
extracting the spent material with ethyl acetate to obtain second carotenoid extract; and
combining together the polysaccharide-containing extract and the first and the second carotenoid-containing extracts.

22. The method of claim 21, further comprising combining the polysaccharide-containing extract and the first and the second carotenoid-containing extracts with at least one pharmaceutically acceptable additive.

23. The method of claim 21, further comprising adjusting the polysaccharide content of the composition to a final concentration ranging from about 10%-about 80% w/w.

24. The method of claim 21, further comprising adjusting the carotenoid content of the composition to a final concentration ranging about 0.10%-about 80% w/w.

25. The method of claim 21, wherein the polysaccharide concentration and carotenoid concentration are adjusted independently of each other.

26. A method of treating a subject with a medical condition by administering to the subject a therapeutically effective amount of a composition comprising polysaccharides and carotenoids extracted from Goji species.

27. The method of claim 26, wherein the medical condition includes any of ocular disorders, diabetes, bone metabolism, arthritis, muscle loss, cardiovascular disorders, immunological disorders, fatigue, obesity, sexual disorders, dermatological disorders, and neurological disorders

28. The method of claim 27, wherein the composition further comprises at least one second agent, wherein the medical condition is an ocular disorder, and wherein the second agent provides a salutory effect with regard to the ocular disorder.
